(19) United States
(12) Patent Application Publication
(10) Pub. No.: US 2021/0244868 A1
(43) Pub. Date: Aug. 12, 2021

(54) APPARATUS FOR BLOOD TREATMENT AND METHOD FOR INITIALIZING SUCH APPARATUS

(71) Applicant: EXTRA CORPOREAL SOLUTIONS S.R.L., Salemo (IT)

(72) Inventor: Michele GAZZANO, Albairate (IT)

(21) Appl. No.: 16/972,631

(22) PCT Filed: Jul. 2, 2019
(86) PCT No.: PCT/IT2019/050158

§ 371 Date: Dec. 7, 2020

(30) Foreign Application Priority Data
Jul. 5, 2018 (IT) 102018000006973

(51) Int. Cl. A61M 1/16; A61M 1/36; A61M 1/26

(52) U.S. Cl. CPC A61M 1/1698; A61M 1/3626; A61M 2205/3337; A61M 2205/3334; A61M 1/267

(57) ABSTRACT

An apparatus for treating blood with veno-venous access comprising: a blood circuit (C1, C2) provided with an inlet branch (C3) and an outlet branch (C4), an oxygenator (OX), a blood pump (P) adapted for drawing blood from the patient and returning it to the patient at a first blood flow value. The blood pump (P) is a volumetric peristaltic pump; the blood circuit (C1, C2) comprises a high-speed portion (C1) and a recirculation circuit (C2); the apparatus (100) comprises at least one pressure measuring means (PA; PR; PP), as well as automatic flow regulation means (FD) acting on the recirculation circuit to determine a second blood flow value in the same recirculation circuit (C2) so that in the high speed portion (C1) the flow value is the sum of said first and second flow values.

The present invention relates to an apparatus for treating the blood and a method for initializing the same apparatus.

More precisely, the invention relates to extracorporeal treatments involving the removal of CO₂.

The aforementioned treatment, like any extracorporeal treatment, necessarily involves an access to the patient through a blood vessel of appropriate size. In the case of the present invention the treatment is of the veno-venous type; the access is practiced by inserting a catheter into a vein such as, for example, the femoral vein, the jugular vein, the carotid vein or others at the discretion of the doctor.

For a generic CO₂ removal treatment, blood is drawn from the vein by means of a catheter, usually two-way type. Thanks to the action of a pump (generally a peristaltic or a centrifugal pump) the blood is pushed into tubes that convey it to a medical device, called oxygenator, which provides to the gas exchange by extracting the CO₂ and administering O₂ thanks to a partial pressure difference through a gas-permeable membrane (generally in Polypropylene or in Polymethylpentene).

Examples of oxygenators are described in U.S. Pat. No. 6,117,390 and EP-0005866. These documents show examples of embodiment of oxygenator devices which comprise a housing provided with inlet and outlet openings for oxygen and blood and a bundle of hollow fibers, which are arranged inside the housing and allow the exchange of O₂ and CO₂. EP-0005866 describes an example of an oxygenator of compact dimensions; U.S. Pat. No. 6,117,390 describes an oxygenator connectable to the patient by means of a venous/arterial connection.

In an extracorporeal treatment, after passing through the oxygenator, the blood is returned to the patient through the same two-way catheter by which it was taken. The circuit, that is, the set consisting of catheter, tubes, active components (such as the oxygenator or others) is completed by accessories such as pressure sensors, flow meters, air bubble sensors, drippers, debuggers, accesses for blood collection and for the administration of drugs, anticoagulants etc., using components known to specialists in the field which for greater clarity and brevity will be omitted in the description of the present invention considering the various components as present where appropriate.

The effectiveness of gas exchange is proportional to the surface of the gas-permeable membrane, (generally expressed in square meters) and to the flow of blood that passes through it, generally expressed in ml/min (milliliters per minute). The higher the flow and the greater the exchange surface, the more effective the extraction of CO₂.

It follows that, limited to the need to insert a catheter into the vein that is as small as possible to achieve the desired effect and to minimize the invasiveness of the therapy, it is the objective of this treatment, commonly called ECCO₂R (Extra Corporeal CO₂ Remover) to obtain the highest possible blood flow inside the oxygenator.

In the present description the term flow or flux is used to indicate the quantity of fluid (i.e. the volume of blood) passing through a section of the duct or in the device in which it moves in the unit of time.

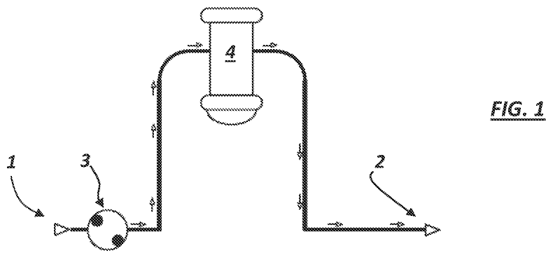

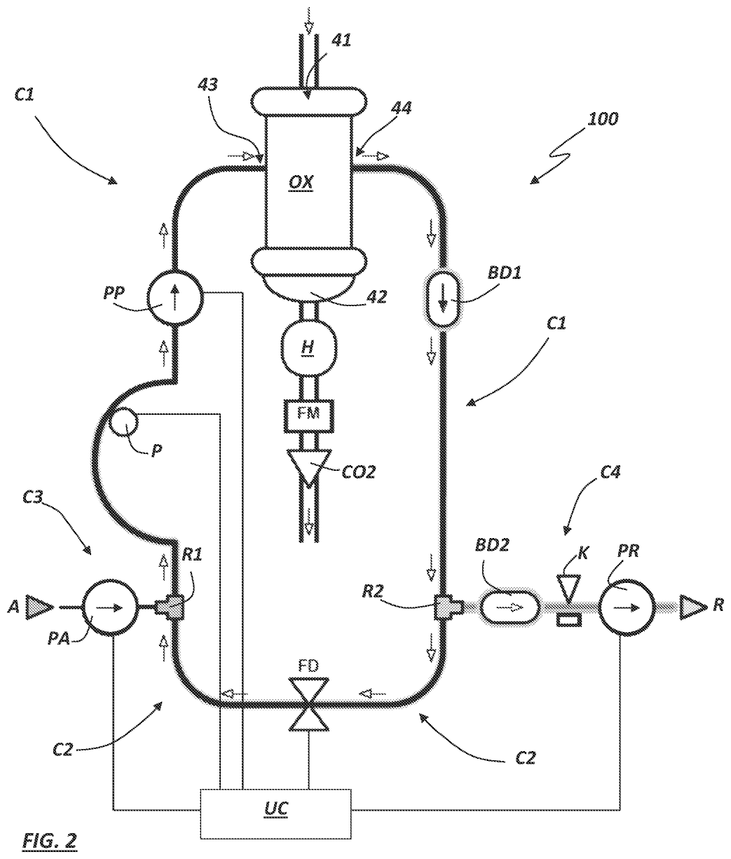

FIG. 1 schematically shows a traditional circuit. FIG. 2 schematically shows the circuit of a blood treatment apparatus according to the present invention in a possible embodiment. In the drawing of FIG. 1, the circuit is connected to the patient by a first access or inlet (1) of the blood circuit to draw the blood to be treated and returns the blood treated to the patient through the second access or exit (2) of the blood circuit. The illustrated circuit comprises a pump (3) and a device (4) for performing a treatment on the blood; the device (4) can be constituted, for example, by an oxygenator. In a traditional circuit, the blood circulating in the circuit passes through the device (4) with a flow determined by the flow rate of the pump (3) and which must be compatible with the flow at which the blood must be drawn and then returned to the patient; in practice, the oxygenator (4) must necessarily be used for the flow with which the blood is drawn and then returned to the patient even if the same device (4) is structurally and functionally configured to work at higher flows, with corresponding higher returns.

Object of the present invention is to make this extracorporeal treatment more efficient allowing, for the same blood flow taken (and therefore returned) to the patient, to make a significantly greater flow circulate through the oxygenator, thus obtaining a better treatment efficiency and other advantages that will be described later.

It will therefore be possible to regulate the blood flow that will be withdrawn (and returned) to the patient and, at the same time, to circulate a higher blood flow in the oxygenator which, as described below, is the sum of the flow taken from the patient and the recirculated flow.

With reference to FIG. 2, this particular characteristic is obtained with a circuit in a “ring” configuration whose connection to the patient is achieved by means of a two-way catheter, with a lumen (A) for the collection of the blood to be treated and a lumen (R) for returning the treated blood. The two lumens (A, R) of the catheter are connected to the blood circuit of the apparatus in correspondence of two fittings (R1, R2) arranged respectively upstream and downstream of the oxygenator (OX), dividing the circuit into a first portion on the which is arranged the oxygenator (OX) and a second portion on which a flow regulator (FD) is arranged. In the attached figures the accesses to the catheter (A) and (R) are schematically represented by triangles that indicate the direction of the flow. On the first portion there is a volumetric blood pump (P) which sucks blood from the access (A) inserted into the patient's vein and sends it to the oxygenator (OX) placed downstream; from the oxygenator (OX) the blood arrives at the second fitting (R2) where a part of it is returned to the patient through the access (R) and another part returns to the first fitting (R1) crossing the flow regulator (FD) which is motorized. In practice, the blood in the apparatus circuit is subjected to the action of the pump (P) and of the motorized flow regulator (FD), being able to differentiate the flow values in order to keep the withdrawal and return flow to the patient at a value and the flow of blood passing through the oxygenator to another value, even considerably higher than the previous one. In other words, the circuit is divided into a main portion or high-speed portion and a secondary portion or recirculation circuit, and is connected to the patient via the access and return lines.

The circuit of the present invention offers numerous advantages, among which the ones described below can be mentioned.

1) It allows, with the same flow rate taken by the patient, to subject a significantly greater flow to the action of the oxygenator (with consequent greater efficiency of CO₂ extraction).

2) It allows, thanks to the greater blood flow in the oxygenator, to use an oxygenator of greater surface (and therefore greater efficiency) without the risk that the excessive slowing of the blood flow in a large surface oxygenator may give rise to stagnation, to creating preferential routes and greater risk of clots.

3) It also allows, independently of the amount of blood flow in the oxygenator, to accurately dose out the blood flow taken (and returned) to the patient.

4) In the case in which, as in the preferred solution in the present invention, the necessary air flow inside the oxygenator is sucked by a pump downstream of the gas circuit of the same oxygenator rather than pushed by a pump upstream or by a connection to a pressure regulator (from a cylinder or a distribution network as found in many hospitals), a negative pressure will be obtained in the gas compartment of the oxygenator. This solution, in addition to avoiding the risk of introducing bubbles in the blood circuit, allows the oxygenator to perform the function of eliminating the bubbles since any gas present in bubbles in the blood circuit is trapped by the fibers of the oxygenator and eliminated from the negative pressure present inside the oxygenator itself. In this case, thanks to the circuit object of the present invention it is also possible to automatically eliminate any bubbles with the method described in the following point 5).

5) Thanks to the conformation of the recirculation circuit, if air bubbles are detected in the blood flow before returning to the patient, in the section of the circuit downstream of the oxygenator and upstream of the connection where the relative sensor will be positioned, it is possible automatically eliminating the bubbles themselves by interrupting the flow to the patient in the section of the circuit indicated with (C4) in the enclosed drawings thanks to a clamp positioned there, and making the blood circulate with the bubbles through the oxygenator.

The case of air bubbles in the blood flow is currently the type of alarm whose solution requires the greatest skill to the operator and often leads to the complete replacement of the circuit with obvious economic damage, loss of time and waste of the patient's blood present in the circuit to be replaced.

6) In the case of temporary interruption of the treatment, for example for the cleaning of the catheter, it allows to keep in circulation the blood inside the oxygenator thus avoiding the risk of clots, simply blocking the flow of blood towards the patient and circulating blood only in the “loop” and in the oxygenator.

7) A further advantage relates to the methods of detecting the flow rate value within the circuit of the present apparatus, a value which, as described below, can be determined simply by measuring the pressure, thus eliminating flow meters (flux-meters) or similar sensors that generally determine a haemolysis and that usually significantly increase production costs.

Further objects and advantages of the present invention will be more evident from the following description, which refers to the attached drawings which constitute a non-limiting embodiment and in which:

FIG. 1 schematically illustrates apparatuses of known type in which the active component (oxygenator or other) is arranged in series along the line;

FIG. 2 schematically illustrates a possible example of embodiment of the invention;

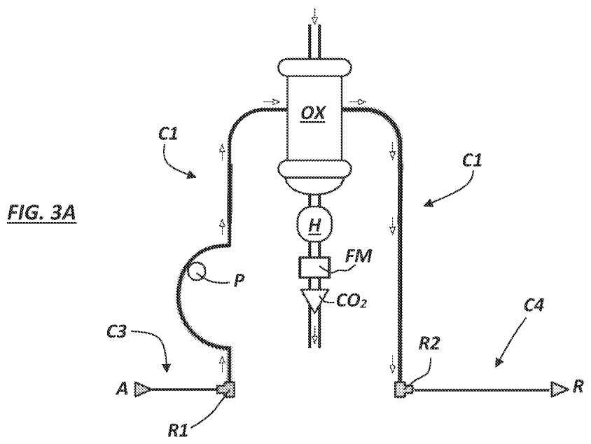

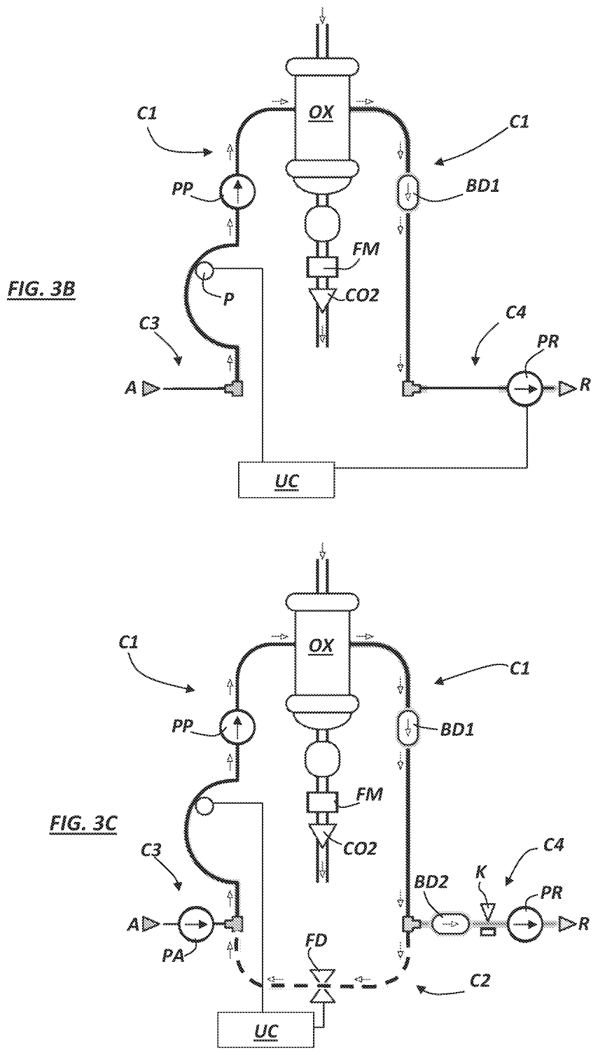

FIGS. 3A, 3B, 3C are schematic representations of successive phases of a possible initialization process of the apparatus object of the invention.

An apparatus (100) according to the invention can be used for extracorporeal blood treatment with veno-venous access and is of the type comprising:
a blood circuit (C1, C2) for the blood to be treated provided with an inlet branch (C3) for the blood to be treated and an outlet branch (C4) for the treated blood, said inlet branch (C3) being provided upstream of an inlet (A) for the blood to be treated, said exit branch (C4) being provided with a downstream outlet (R) for the treated blood, said inlet (A) and said outlet (R) being provided with veno-venous connection means to the patient, preferably by means of a double lumen catheter;
an oxygenating device (OX) able to perform a treatment on the blood, arranged and acting on said circuit (C1, C2);
pumping means comprising a blood pump (P) acting on said circuit (C1, C2), apt to move blood with upstream-to-downstream direction and adapted to draw blood from the patient and return it to the patient at a first value of blood flow.

The apparatus (100), which is provided with means of veno-venous connection to the patient, is characterized in that:
said blood pump (P) is a volumetric peristaltic pump;
said blood circuit (C1, C2) comprises a main portion or high-speed portion (C1) and a secondary portion which defines a recirculation circuit (C2); said high-speed portion (C1) being connected upstream to a first fitting (R1), connected to said inlet branch (C3), and being connected downstream to a second fitting (R2), connected upstream of said branch exit (C4); said secondary portion (C2) being connected upstream to said second fitting (R2) and downstream to said first fitting (R1);
said oxygenator device (4) is arranged and acts on said main portion or high speed portion (C1);
the apparatus (100) comprises at least one flow measurement means (PA; PR) acting on at least one among said inlet branch (C3) and said outlet branch (C4);
the equipment (100) includes automatic and motorized flow regulation means (FD) acting on the recirculation circuit (C2);
the apparatus (100) comprises a central processing unit (UC) connected to said pump (P) and to said motorized flow regulation means (FD) as well as to said at least one flow measurement means (PA; PR), capable of automatically controlling said flow adjustment means (FD) to determine in said recirculation circuit (C2) a second blood flow value so that in said high speed portion (C1) the flow value is the sum of the said first and second blood flow values.

As previously expressed, the apparatus is provided with a volumetric peristaltic pump (P) arranged on the high speed portion (C1): for this reason the blood flow is determined by the rotation of the pump (P).

In the present description the definitions “upstream” and “downstream” refer to the path followed by the blood in the apparatus (100). The arrows indicate the direction followed by the blood in the apparatus (100).

In the present description, the high-speed portion (C1) is also called main portion (C1); the recirculation circuit (C2) is also called secondary portion (C2).

The inlet (C3) and outlet (C4) branches are preferably supported by a single double lumen venous catheter.

In particular, the aforementioned at least one means of measuring the flow (PA; PR) can advantageously be a pressure meter suitable for measuring the pressure value within the branch (C3; C4) on which it is acting. In practice, as will be better described below, with the present apparatus it is possible to obtain the value of the blood flow in the circuit by measuring its pressure. Pressure measurement with non-hemolytic instruments significantly improves the impact of the apparatus on the patient's blood. For this purpose, known type of pressure meters and therefore not described in detail can be used.

As in the example of FIG. 2, third flow measurement means (PP) can be provided downstream of said pump (P) and upstream of said oxygenator (OX).

Also in this case, the flow measuring means (PP) can advantageously be a pressure meter suitable for measuring the pressure value within the branch (C1) on which it is acting.

The detection of the flow measurement means (PP), in the version in which they are pressure measurers, can be used to limit the flow rate of the pump (P) so that the value of the detected pressure remains below a preset maximum pressure value; the variation over time of this measured value, with the same pump flow rate (P), will provide valid indications on the fluidity of the blood during treatment and on the possible need to change the amount of anticoagulant.

Advantageously, the first flow value (that is the value of the blood flow in branches C3 and C4 which, respectively, draw blood from the patient and return it to the same patient) can be between 0 (zero) and 500 ml/min and the flow value in the main portion (C1) can be between 0 (zero) and 2000 ml/min. In practice, in the recirculation circuit (C2) the flow will be equal to the difference between the flow of the high speed portion (C1) and the flow in the branches (C3, C4) for the blood collection and return to the patient.

The apparatus can comprise a means for closing the blood flow (K) (or clamp), arranged downstream of said second fitting (R2) and upstream of said outlet (R). Furthermore, it can comprise at least one means for detecting the presence of air bubbles in the blood (BD1, BD2) able to activate said means for closing the flow (K) in correspondence of the detection of bubbles in the circuit.

In practice, the apparatus (100), in the preferred form illustrated in FIG. 2, is advantageously provided with automated means for closing the flow (K), driven by the central unit (UC), which is able to block the flow of blood in case of detection of air bubbles by the detectors (BD1) and (BD2). The automated means for closing the flow (K) and the detectors (BD1) and (BD2), which may consist respectively of clamps or valves and of bubble detectors, are devices known per se and therefore not described in detail. The connection between the bubble detectors (BD1, BD2), the central unit (UC) and the flow closure means (K) is not graphically represented in the drawings.

In the example of FIG. 2 the pump (P) is arranged and acting upstream with respect to the oxygenator (OX). In this way the blood is under pressure inside the oxygenator, preventing air bubbles from entering it and indeed facilitating its elimination. The possible positioning of the pump downstream of the oxygenator would not provide the same advantages.

Advantageously, the present apparatus can comprise an air suction pump (H) connected to the oxygenator (OX) at a gas outlet (42) of the latter so that the flow of gas determined inside the oxygenator (OX) consists of a downstream suction with negative pressure in the gas compartment of the oxygenator. In the drawings, the oxygenator (OX) is provided with a gas inlet (41), a gas outlet (42) on which the air/oxygen pump (H) acts, as well as a blood inlet (43) and a blood outlet (44). Furthermore, with the rectangular block (FM) an air flow meter is schematically represented and a triangular block (CO₂) represents a sensor of the CO₂ extracted from the blood. The air flow meter (FM) and the sensor (CO₂) are appropriately connected to the control and command means of the equipment.

The oxygenator (OX) can be of the gas-permeable membrane type (for example in Polypropylene or in Polymethylpentene) with an exchange surface of between 0.4 m² (for pediatric use) up to 1.5 m² and more (for high efficiencies). The characteristics and component parts of the oxygenator are not described in detail since they are of a type known to technicians in the sector.

With the apparatus in operation, the blood taken from the patient by the access (A) is introduced into a ring circuit composed of the high-speed portion (C1) and the recirculation circuit (C2), connected to each other at the two fittings (R1) and (R2) that define the connections with the inlet (A) and the outlet (R), i.e. with the two-lumen catheter connected to the patient.

The flow regulation means (FD) can advantageously be constituted by a clamp regulated by a servomotor controlled by the central unit (UC) capable of reducing and possibly blocking the flow of blood in the section of conduct subjected to its action. In other words, the servomotor of the flow regulation means (FD) under command of the central unit (UC) determines the opening of the secondary portion (C2), with an activation of the clamp of the means (FD) which can advantageously be variable and gradual.

The flow regulation means (FD) are shaped so as to determine in the main portion (C1) a flow of maximum value, the value of this flow being equal to the sum of the first flow value (which is the flow with which the blood is taken from the patient and returned to the patient himself) and of the second flow value (which is the flow determined by the regulator FD in the secondary portion C2). In particular, the said maximum flow value maintained in the high speed portion (C1) corresponds to the flow determined by the pump flow rate (P).

In practice, the flow of blood taken (and returned) to the patient is determined by the value of the flow exerted by the pump (P) reduced by the part related to the flow regulator (FD). In the oxygenator (OX) passes a blood flow determined by the flow rate of the pump (P), a flow that would not be feasible with a known type of device in which the oxygenator must be traversed by the same blood collection/return flow.

In other words, the flow value in the portion (C2) (or second value) is equal to the difference between the maximum flow value generated by the pump (P) and the first flow value taken and returned to the patient, as well as the value of maximum flow in the high speed portion (C1) is equal to the sum of the flow withdrawn (and returned) to the patient (first value) and of the flow circulating in the secondary portion (C2), i.e. to the value of the flow generated by the single pump (P) and subjected to the action of the regulator (FD).

As an example and coherently with the example shown in FIG. 2, the blood pump (P) (which is the only pump in the circuit) pushes 2000 ml/min of blood, that is determines a flow of 2000 ml/min; this flow is composed of the sum of the 500 ml/min taken by the patient from the access point (A), resulting from the relationship between pressure and flow rate relative to the pressure meter (PA) downstream of the access point (A) to the patient (or from the PR meter upstream of the return point R) and from the 1500 ml/min that the flow regulator (FD) lets pass in the recirculation circuit.

Referring again to the illustrated examples, the apparatus of the invention comprises a blood circuit which is suitably connected to a patient to take the blood to be treated and return the treated blood. In the drawings it is marked with (A) the entrance of the blood to be treated and with (R) the downstream exit that returns to the patient the blood that has been treated. The two accesses (A) and (R), as previously mentioned, may consist of a double lumen catheter.

This solution advantageously allows to carry out the invention thanks to a single blood pump with evident economic advantages and of treatment quality thanks to the greater efficiency in the removal of CO₂, to the lower formation of coagula inside the oxygenator and, thanks to the circulation of gas in depression inside the oxygenator, also the automatic elimination of air bubbles possibly present inside the blood circuit.

The operation of the equipment object of the invention is particularly effective also on the basis of the initialization method that will be described below. In particular, it is extremely advantageous to measure the pressure instead of the flow rate compared to apparatuses of the known type.

For example, in apparatuses of the known type, i.e. in the case of circuits that use centrifugal pumps (or in any case non-volumetric pumps) it is necessary to use a flow meter since the operating speed of the pump cannot be linearly correlated with the flow rate that it generates since this flow rate is a function not only of the pump speed, but also of the resistances that the generated flow encounters.

In the present invention, with the use of a volumetric (peristaltic) pump, the flow meter is not necessary since the flow rate of the pump according to the operating speed is always known with sufficient precision. In other words, the speed of the pump substantially determines the value of the blood flow in the circuit.

The preferred version of the present invention, however, needs to know the data relating to the flow rate since the same, although known since it is generated by a volumetric pump, is subdivided into several portions of circuit of which at least that which comprises the withdrawal branches and/or of blood return to the patient needs a flow control.

In the present invention, we can advantageously use at least one of the pressure sensors (PA, PR) to correctly deduce the flow and therefore the flow rate in the concerned section of circuit.

A possible initialization method, schematically illustrated in FIGS. 3A-3C, can include the following steps:

a) Closure of the recirculation circuit (C2); in this phase the flow regulator (FD), i.e. the relative servomotor which regulates the circuit opening value (C2) and which can completely obstruct the same recirculation circuit, is completely closed thus interrupting the recirculation; in other words, the connection from the fitting (R2) to the fitting (R1) is interrupted, with a situation that can be schematically represented by the drawing in FIG. 3A.

b) Pump activation (P); in this phase the volumetric pump (P) is activated, which starts from zero and, gradually increasing its flow rate, reaches a value of maximum pre-set pressure or test value that is detected by the pressure sensor (PA) and/or (PR), as schematically represented in FIG. 3B where only the sensor (PR) is represented.

c) Memorization of the measured pressure values and their combination with the corresponding flow values; during this operation the electronic circuit, which is represented schematically by the block UC, records and stores the correspondence of values between the flow rate (which is determined by the speed of the pump P) and the pressure relative to the flow rate itself, detected by the sensor (PA) and/or (PR). It can therefore be established that for each pressure value there is a specific flow (if there are no variations on the circuit).

d) Gradual increase in pump speed up to a maximum working pressure value; in this phase the speed of the pump (P) is gradually increased up to its maximum value and therefore its flow rate (corresponding to about 2.000 ml/min) while the flow regulator valve (FD) is gradually opened (this situation is schematically represented by the secondary portion C2 which in FIG. 3C is represented in a discontinuous line); the (FD) valve, controlled by the electronic circuit (UC) that manages its driving, will open in such a way as to maintain the pressure detected by the sensor so that it corresponds to the desired flow (or flow rate).

In this way it is possible not to use a flowmeter, which having to be non-haemolytic generally works with Doppler effect or taking advantage of the Coriolis acceleration and usually has a considerable cost.

e) Repetition of phases a) to d) at determined intervals. Given the possibility that changes occur on the characteristics of the circuit (changes in the fluidity of the blood, partial obstructions of the catheter, etc.) the calibration of the pump/pressure sensor assembly will be repeated at predetermined intervals (for example every 5 min.)

The pressure sensor (PP) upstream of the oxygenator will limit the maximum speed of the pump (P) when the read pressure value reaches a preset alarm value.

Furthermore, when at the same speed of the pump (P) the sensor (PP) will detect a tendency to increase the pressure, it will provide an alarm signal to the operator relative to the fact that the fluidity of the blood is decreasing and that it could be it is appropriate to increase the administration of anticoagulant.

In practice, the continuous feedback between the pressure sensors (PA, PR), the regulation of the pump flow (P) and the opening of the regulator valve (FD) make the system very automated, reducing the need for manual interventions and for at the same time they allow to reach high efficiencies of CO₂ removal in total safety and with reduced invasiveness.

Naturally, the invention is not limited to what has been described and illustrated, but can be widely varied as regards the arrangement and the nature of the components used without thereby abandoning the inventive teaching set forth above and claimed below.

1. An apparatus for extracorporeal blood treatment with veno-venous access of the type comprising:
a blood circuit for the blood to be treated provided with an inlet branch for the blood to be treated and an outlet branch for the treated blood, said inlet branch being provided upstream of an inlet for the blood to be treated, said exit branch being provided with a downstream outlet for the treated blood, said inlet and said outlet being provided with veno-venous connection means by catheter to the patient;
an oxygenating device adapted to perform a treatment on the blood, arranged and acting on said circuit;
pumping means comprising a blood pump acting on said circuit, adapted to move blood in upstream-to-downstream direction and adapted to draw blood from the patient and return it to the patient at a first value of blood flow;
wherein:
said blood pump is a volumetric peristaltic pump;
said blood circuit comprises a high-speed portion and a recirculation circuit; said high-speed portion being connected upstream to a first fitting, connected to said inlet branch, and being connected downstream to a second fitting, connected upstream to said outlet branch; said secondary portion being connected upstream to said second fitting and downstream to said first fitting;
said oxygenator device is arranged and acts on said high speed portion;
the apparatus comprises at least one flow measurement means acting on at least one among said inlet branch and said outlet branch;
the apparatus comprises automatic and motorized flow regulation means acting on said recirculation circuit;
the apparatus comprises a central processing unit connected to said pump and to said flow regulation means as well as to said at least one flow measurement means, adapted to automatically control said flow regulation means to determine in said recirculation circuit a second blood flow value so that in said high speed portion the flow value is the sum of said first and second flow value.

2. The apparatus of claim 1, wherein said at least one flow-measuring means is a pressure meter suitable for measuring the pressure value inside the branch on which it is agent.

3. The apparatus of claim 1, wherein said pump is arranged upstream of said oxygenator.

4. The apparatus according to of claim 1, wherein said pump is arranged upstream of said oxygenator and the apparatus comprises an additional flow measurement means (PP) arranged on said high speed portion (C1) downstream of said pump (P) and upstream of said oxygenator (OX).

5. The apparatus of claim 1, wherein said pump is arranged upstream of said oxygenator and the apparatus comprises an additional pressure measurement means (PP) arranged on said high speed portion (C1) downstream of said pump (P) and upstream of said oxygenator (OX).

6. The apparatus of claim 1 wherein said first flow value is between 0 and 500 ml/min and the flow value in the high-speed portion (C1) is between 0 and 2000 ml/min.

7. The apparatus of claim 1 wherein it comprises a means for closing the flow of blood arranged and acting on said output branch, downstream of said second fitting and upstream of said outlet.

8. The apparatus of claim 1 wherein it comprises a means for closing the flow of blood arranged and acting on said output branch, downstream of said second fitting and upstream of said outlet and wherein it comprises at least one means for detecting the presence of air bubbles in the blood adapted to activate said means for closing the flow at a bubble detection in the circuit.

9. The apparatus of claim 1 wherein it comprises an air pump connected to said oxygenator at a gas outlet of the latter so that the gas flow determined inside of the oxygenator is a downstream suction with negative pressure in the gas compartment of the oxygenator.

10. A method for initializing an apparatus for extracorporeal blood treatment with veno-venous access of the type comprising:
a blood circuit for the blood to be treated provided with an inlet branch for the blood to be treated and an outlet branch for the treated blood, said inlet branch being provided upstream of an inlet for the blood to be treated, said exit branch being provided with a downstream outlet for the treated blood, said inlet and said outlet being provided with veno-venous connection means by catheter to the patient;
an oxygenating device adapted to perform a treatment on the blood, arranged and acting on said circuit;
pumping means comprising a blood pump acting on said circuit, adapted to move blood in upstream-to-downstream direction and adapted to draw blood from the patient and return it to the patient at a first value of blood flow;
wherein:
said blood pump is a volumetric peristaltic pump;
said blood circuit comprises a high-speed portion and a recirculation circuit; said high-speed portion being connected upstream to a first fitting, connected to said inlet branch, and being connected downstream to a second fitting, connected upstream to said outlet branch; said secondary portion being connected upstream to said second fitting and downstream to said first fitting;
said oxygenator device is arranged and acts on said high speed portion;
the apparatus comprises at least one flow measurement means acting on at least one among said inlet branch and said outlet branch;
the apparatus comprises automatic and motorized flow regulation means acting on said recirculation circuit;
the apparatus comprises a central processing unit connected to said pump and to said flow regulation means as well as to said at least one flow measurement means, adapted to automatically control said flow regulation means to determine in said recirculation circuit a second blood flow value so that in said high speed portion the flow value is the sum of said first and second flow value,
wherein the method comprises the following steps:
a) closing the recirculation circuit;
b) activation of the pump and gradual increase of its speed until reaching a value of maximum pre-set value pressure or test pressure detected by said at least one means of flow measurement;
c) memorization of the pressure values detected by said at least one flow measurement means and their combination with the corresponding pump flow rate values;
d) gradual increase in pump speed up to a maximum flow rate value corresponding to a predetermined maximum working pressure value detected by said at least one flow measuring means.

11. The method of claim 10, wherein said maximum flow rate value is equal to about 2000 ml/min.

12. The method of claim 10 wherein it comprises the repetition of steps a) to d) at determined intervals.

13. The method of claim 10 wherein said maximum flow rate value is equal to about 2000 ml/min and wherein it comprises the repetition of steps a) to d) at determined intervals.
